(19) United States
(12) Patent Application Publication
(10) Pub. No.: US 2011/0144354 A1
(43) Pub. Date: Jun. 16, 2011

(54) Process for Preparation of Darifenacin and Intermediates Used in the Process

(75) Inventors: Manjunath Narayan Bhanu, Pune (IN); Samir Naik, Thane (IN); Arjun Bodkhe, Ulhasnagar (IN); Amit Soni, Maharashtra (IN)

(73) Assignee: WATSON PHARMA PRIVATE LIMITED, Andheri (E), Mumbai (IN)

(21) Appl. No.: 13/060,150

(22) PCT Filed: Sep. 21, 2009
(86) PCT No.: PCT/IB09/54138

§ 371 Date: Feb. 22, 2011

(30) Foreign Application Priority Data
Sep. 22, 2008 (IN) 2046/MUM/2008
Sep. 21, 2009 (IB) PCT/IB2009/054138

(51) Int. Cl. C07D 207/14

(52) U.S. Cl. 548/531

(57) ABSTRACT

Disclosed herein is a process for the preparation of darifenacin and physiologically acceptable salts thereof. Also disclosed is a compound of formula X: wherein R is linear or branched C₁₋₁₀ alkyl, phenyl, tolyl, ortho-, meta- or para-xylyl which is useful as a intermediate in the preparation of darifenacin.

### TECHNICAL FIELD OF THE INVENTION

The present invention relates to an improved process for preparing physiologically acceptable salts of darifenacin and intermediates useful in the production of darifenacin. The physiologically acceptable salts are preferably acid addition salts, such as hydrobromide, hydrochloride and hydroiodide.

### BACKGROUND OF THE INVENTION

Darifenacin, also known as (S)-2-{1-[2-(2,3-dihydrobenzofuran-5-yl)ethyl]-3-pyrrolidinyl}-2,2-diphenylacetamide and shown below as formula I, is a selective M3 receptor antagonist used for the treatment of overactive bladder.

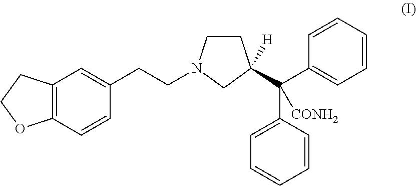

Darifenacin, marketed under the tradename ENABLEX®, is administered as the hydrobromide salt, (S)-2-{1-[2-(2,3-dihydrobenzofuran-5-yl)ethyl]-3-pyrrolidinyl}-2,2-diphenylacetamide hydrobromide shown below as formula II

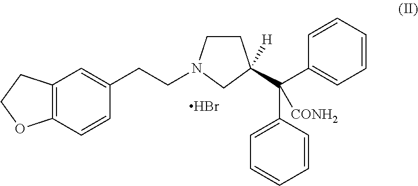

Darifenacin is disclosed in U.S. Pat. No. 5,096,890 (the '890 patent), which describes various routes for the synthesis of darifenacin hydrobromide. Each of the synthetic routes disclosed within the '890 patent recite the hazardous Mitsunobu reaction, as described in Scheme I below.

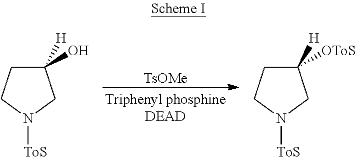

Within the reaction set forth in Scheme I, 1-toyl-3-(R)-pyrrolidinol is reacted with methyl tosylate and diethylazodicarboxylate (DEAD). The product obtained is contaminated with triphenylphosphine oxide, which is difficult to separate from the desired product. Moreover, other toxic and hazardous reagents, such as pyridine and sodium hydride, are used in other steps of the synthesis.

U.S. Patent Publication No. 2003/0191176 discloses a process for the synthesis of darifenacin that requires the use of boron trifluoride and carbonyl diimidazole, both of which are toxic reagents.

U.S. Patent Publication No. 2007/0197631 (“the '631 publication”) describes the preparation of darifenacin via darifenamine using NaBH₄, LiAlH₄ and H₂/Pd as reducing agents, all of which are hazardous to handle on a commercial scale. The process described within the '631 publication also involves the use of sodium tertiary butoxide, which is also potentially hazardous during commercial manufacturing. The '631 publication further describes the synthesis of darifenacin from 3-(S)-(+)-hydroxypyrrolidine, comprising:
i. combining 3-(S)-(+)-hydroxypyrrolidine, a sulfonyl halide, and a base in a suitable solvent to obtain 1-X-sulfonyl-3-(S)-(−)-X-sulfonyloxypyrrolidine (III);
ii. reacting 1-X-sulfonyl-3-(S)-(−)-X-sulfonyloxypyrrolidine (III), diphenylacetonitrile and an inorganic base in an organic solvent to obtain (S)-2,2-diphenyl-2-(1-X-sulfonyl-3-pyrrolidinil)acetonitrile (IV);
iii. reacting (S)-2,2-diphenyl-2-(1-X-sulfonyl-3-pyrrolidinil)acetonitrile (IV), a bromine acceptor selected from the group consisting of phenol and naphthol and an acid to obtain (S)-2,2-diphenyl-2-(3-pyrrolidinil)acetonitrile salt (V);
iv. combining (S)-2,2-diphenyl-2-(3-pyrrolidinil)acetonitrile salt (V), 2(2,3-dihydrobenzofuran-5-yl)ethylchloride (VI) and a base in a suitable solvent to obtain (S)-2-{1-[2-(2,3-dihydrobenzofuran-5-yl)ethyl]-3-pyrrolidinyl}-2,2-acetonitrile (VII); and
v. converting (S)-2-{1-[2-(2,3-dihydrobenzofuran-5-yl)ethyl]-3-pyrrolidinyl}-2,2-acetonitrile (VII) to darifenacin hydrobromide.
   This synthesis is represented below in Scheme II.

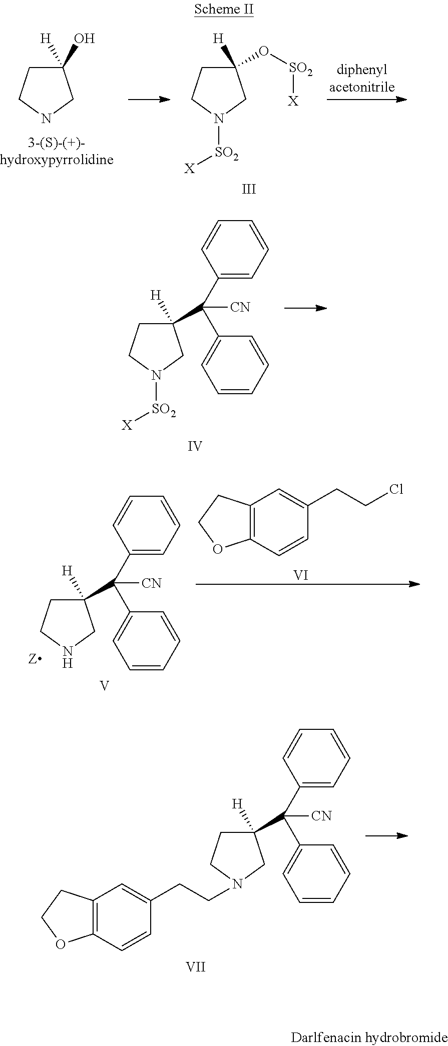

Though the above-described synthesis is potentially feasible on a commercial scale, the darifenacin obtained from the synthesis is of a low purity. Also, the deprotection (detosylation) of the intermediate (S)-2,2-diphenyl-2-(1-X-sulfonyl-3-pyrrolidinil)acetonitrile (IV) is done in aqueous HBr at a temperature of 120° C., which is also a potential hazard.

Therefore, there is a need in the art for a process for the preparation of darifenacin and physiologically acceptable salts thereof such as the hydrobromide that does not use toxic and dangerous reagents and is feasible on a commercial scale. The present invention provides such a process.

### SUMMARY OF THE INVENTION

The present invention provides a process for preparing darifenacin and physiologically acceptable salts thereof such as hydrobromide, hydrochloride and hydroiodide using compounds of formula VIII, which are easily available on commercial scale.

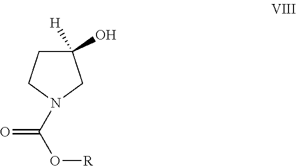

wherein R is linear or branched C₁₋₁₀ alkyl, phenyl, tolyl, ortho-, meta- or para-xylyl. The invention encompasses a process for preparing darifenacin and physiologically acceptable salts thereof comprising:
a) reacting a compound of formula VIII with a sulfonyl halide such as p-toluene sulfonyl chloride and a base in a solvent such as a solvent selected from the group consisting of a C₆₋₉ aromatic hydrocarbon, a polar aprotic organic solvent, water and mixtures thereof to obtain compounds of formula IX

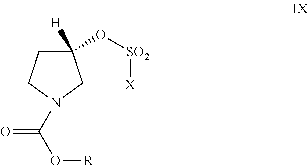

wherein R is linear or branched C₁₋₁₀ alkyl, phenyl, tolyl, ortho-, meta- or para-xylyl and X is linear or branched C₁₋₁₀ alkyl, phenyl, tolyl, ortho-, meta- or para-xylyl;
b) reacting compounds of formula IX, diphenylacetonitrile and an inorganic base in a solvent such as a solvent selected from the group consisting of a C₆₋₉ aromatic hydrocarbon, a polar aprotic organic solvent, water and mixtures thereof to obtain compounds of formula X

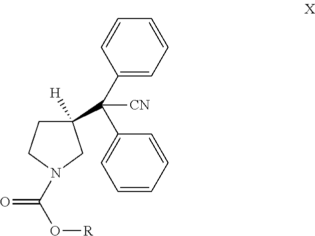

wherein R is linear or branched C₁₋₁₀ alkyl, phenyl, tolyl, ortho-, meta- or para-xylyl;
c) reacting compounds of formula X with a deprotecting agent such as trifluoroacetic acid, tetrabutyl ammonium fluoride or mixtures thereof to obtain (S)-2,2-diphenyl-2-(3-pyrrolidinil)acetonitrile of formula XI

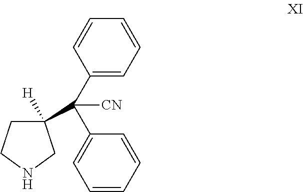

d) reacting (S)-2,2-diphenyl-2-(3-pyrrolidinil)acetonitrile of formula XI with a hydrolyzing agent such as sulfuric acid, hydrochloric acid, an aqueous solution of sodium hydroxide, potassium hydroxide or mixtures thereof to obtain 3-(S)-(+)-(1-carbamoyl-1,1-diphenylmethyl)pyrrolidine of formula XII.

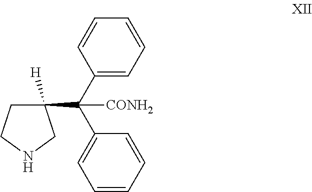

Alternately, 3-(S)-(+)-(1-carbamoyl-1,1-diphenylmethyl)pyrrolidine of formula XII may be prepared directly form compounds of formula X by reacting compounds of formula X with a strong acid such as 95% sulfuric acid to give 3-(S)-(+)-(1-carbamoyl-1,1-diphenylmethyl)pyrrolidine of formula XII.
e) reacting 3-(S)-(+)-(1-carbamoyl-1,1-diphenylmethyl)pyrrolidine of formula XII with an organic acid such as L(+)-tartaric acid, d-malic acid, 1-malic acid, d-mandelic, 1-mandelic acid or mixtures thereof in a solvent such as a solvent selected from the group consisting of alcohols, halogenated hydrocarbons, ketones, water or mixtures thereof to get 3-(S)-(+)-(1-carbamoyl-1,1-diphenylmethyl)pyrrolidine salt of formula XIII

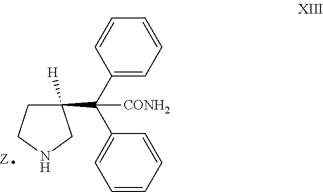

wherein Z is an acid, preferably an organic acid such as L(+)-tartaric acid, d-malic acid, 1-malic acid, d-mandelic, 1-mandelic acid or mixtures thereof;
f) reacting 3-(S)-(+)-(1-carbamoyl-1,1-diphenylmethyl)pyrrolidine salt of formula XIII and a compound of formula XIV

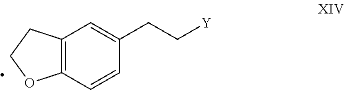

wherein, Y is a leaving group preferably selected from the group consisting of I, Cl, Br, brosyl, tosyl, trifluoroacetyl, in a solvent such as a solvent selected from the group consisting of a C₆₋₉ aromatic hydrocarbon, a polar aprotic organic solvent, water and mixtures thereof and an inorganic base to obtain darifenacin base of formula I

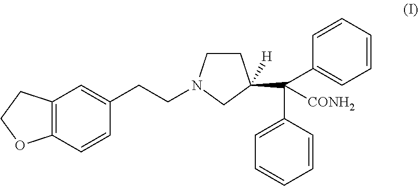

and
g) reacting darifenacin base of formula I with a pharmaceutically acceptable acid or salt such as an aqueous organic acid or inorganic acid selected from the group consisting of hydrobromic acid, hydrochloric acid, hydroiodidic acid, citric acid, formic acid, maleic acid, acetic acid, succinic acid, tartaric acid, methansulphonic acid or toluenesulphonic acid. The reaction may occur in a solvent selected from the group consisting of alcohols, ketones and water or mixtures thereof to obtain the desired physiologically acceptable salts of darifenacin, such as darifenacin hydrobromide of formula II shown above.

The above described process is generally described by Scheme III, shown below.

One embodiment of the present invention also includes a purification step for the crude darifenacin base that involves reducing the amount of unreacted 3-(S)-(+)-(1-carbamoyl-1,1-diphenylmethyl)pyrrolidine in the darifenacin base so the level of the unreacted 3-(S)-(+)-(1-carbamoyl-1,1-diphenylmethyl)pyrrolidine is about 0.05% to about 0.5%. One embodiment of this purification step involves converting crude darifenacin base to its acetate salt by treating with acetic acid. The acetate salt is then converted to the hydrochloride salt in situ by treating with aqueous hydrochloric acid to a pH of about 1 to about 3. The aqueous solution is then extracted with a suitable organic solvent such as methylene chloride and washed to remove the unreacted 3-(S)-(+)-(1-carbamoyl-1,1-diphenylmethyl)pyrrolidine so the level of the unreacted 3-(S)-(+)-(1-carbamoyl-1,1-diphenylmethyl)pyrrolidine in the darifenacin base is about 0.05% to about 0.5%.

The present invention further encompasses a process for the purification of the darifenacin physiologically acceptable salts thereof such as the hydrobromide. The purification of the darifenacin physiologically acceptable salt comprises of dissolving the crude darifenacin salt in 1 to 10 volumes of a first organic solvent such as an alcohol selected from the group consisting of methanol, ethanol, isopropanol or mixtures thereof, at reflux temperature, adding a decolorizing agent and filtering to get a clear filtrate. The clear filtrate is concentrated, and 5 to 20 volumes of a second organic solvent such as acetone is added to the concentrated filtrate and the pure darifenacin salt, having purity by HPLC of 98.5% or higher, preferably 99.0% or higher and most preferably 99.5% or higher is isolated.

Alternatively, the purification of the darifenacin physiologically acceptable salt can be accomplished by dissolving the crude darifenacin salt, such as the hydrobromide salt, in 5 to 25 volumes of water at reflux temperature, adding a decolorizing agent and filtering to get a clear solution, cooling this clear filtrate to about 10° C. to about 40° C., preferably about 15° C. to about 35° C. and most preferably about 20° C. to about 30° C. and isolating pure darifenacin salt having purity by HPLC of more than 98.5%, preferably more than 99.0% and most preferably more than 99.5%.

The present invention also includes the intermediates used in the above described process for preparing darifenacin, such as the compound of formula X and methods for preparing the intermediates, including a method for preparing the compound of formula X.

### DETAILED DESCRIPTION OF THE INVENTION

One aspect of the present invention is to provide a process for the synthesis of darifenacin and physiologically acceptable salts thereof such as the hydrobromide salt of formula II, which can be prepared within the scope of this invention as described above and generally illustrated in Scheme III shown below wherein R and X are as previously described:

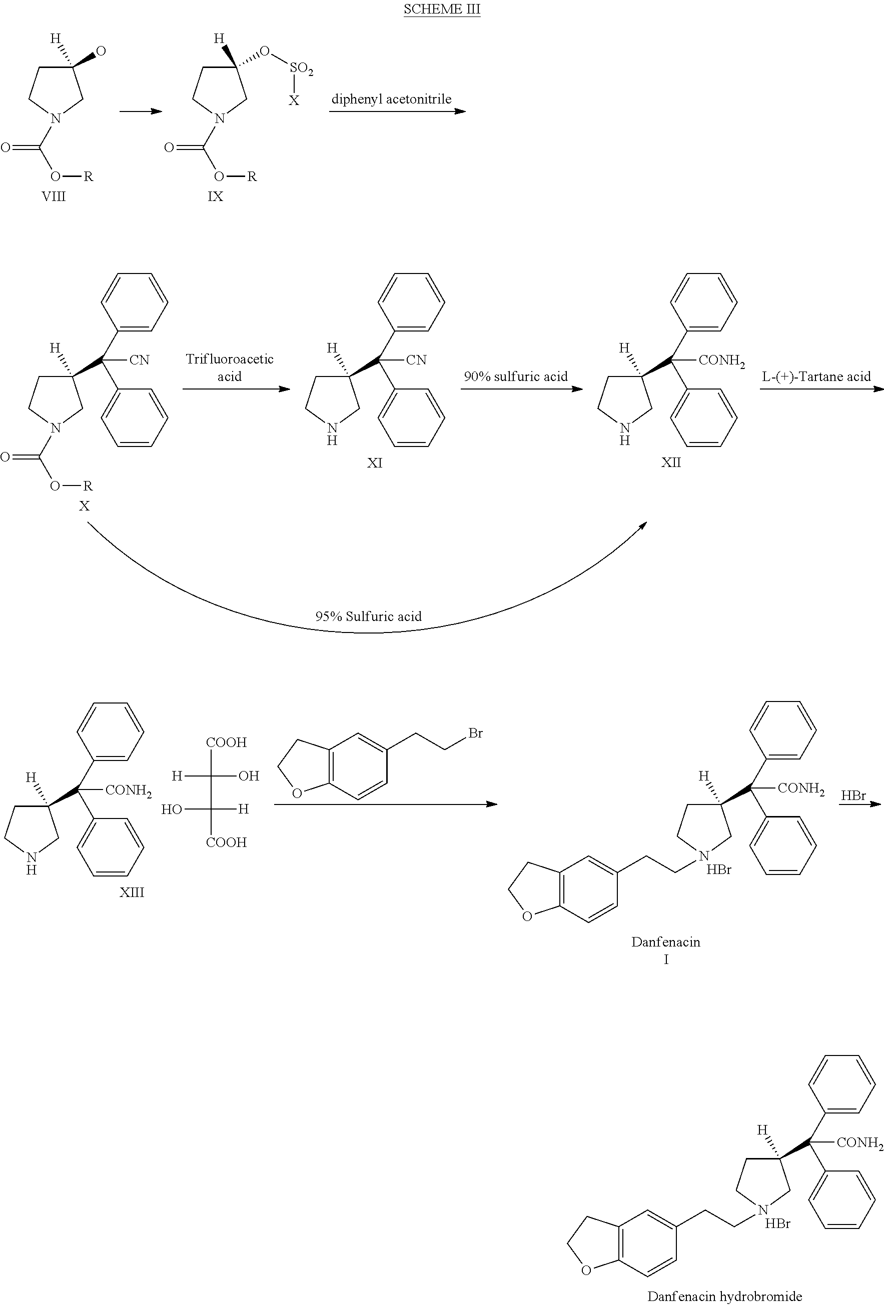

As shown by SCHEME III, one embodiment of the present invention involves the preparation of a compound of formula IX by reacting the compound of formula VIII with a sulfonyl halide, such as p-toluene sulfonyl chloride, in the presence of a phase transfer catalyst such as tetrabutyl ammonium bromide, tetrabutyl ammonium iodide, tetrabutyl ammonium hydroxide or mixtures thereof. The reaction preferably is conducted in a biphasic reaction medium consisting of water and an organic solvent such as toluene, xylene, tetrahydrofuran or mixtures thereof, in the presence of alkali metal salts such as sodium hydroxide, potassium hydroxide, potassium carbonate or mixtures thereof, at a reaction temperature of about 20° C. to about 80° C., preferably about 30° C. to about 70° C. and more preferably about 50° C. to about 60° C. The product of formula IX can be isolated and purified by conventional means if desired.

The compound of formula IX may then be reacted with diphenylacetonitrile to produce a compound of formula X. The reaction of a compound of formula IX and diphenylacetonitrile may be conducted in the presence of a phase transfer catalyst such as tetrabutyl ammonium bromide, tetrabutyl ammonium iodide, tetrabutyl ammonium hydroxide or mixtures thereof in an aqueous medium and an inorganic base such as sodium hydroxide, potassium hydroxide or mixtures thereof. The aqueous concentration of the inorganic base for the reaction is preferably between about 25% w/v to about 70% w/v, preferably between about 30% w/v to about 65% w/v and more preferably between about 40% w/v to about 60% w/v. The reaction is carried out at a temperature range of about 60° C. to about 110° C., preferably at a range of about 75° C. to about 100° C. and more preferably at a range of about 90° C. to about 95° C. The product of formula X can be isolated by conventional means such as extraction into a suitable solvent such as toluene, ethylacetate, methylene chloride or mixtures thereof and further concentrated and crystallized if desired.

Once the compound of formula X is formed and/or isolated, it may be reacted with a deprotecting agent such as an acid selected from the group consisting of trifluoroacetic acid, methane sulfonic acid, tetrabutyl ammonium fluoride or mixtures thereof at a temperature range of about 15° C. to about 60° C., preferably about 20° C. to about 45° C. to produce (S)-2,2-diphenyl-2-(3-pyrrolidinil) acetonitrile of formula XI.

The (S)-2,2-diphenyl-2-(3-pyrrolidinil) acetonitrile of formula XI obtained may be reacted in presence of a hydrolyzing agent such as an aqueous acid to produce 3-(S)-(+)-(1-carbamoyl-1,1-diphenyl methyl)pyrrolidine of formula XII. The hydrolyzing agent may be an aqueous acid such as sulfuric acid or hydrochloric acid or an aqueous base such as sodium hydroxide or potassium hydroxide. If an aqueous acid is employed in this reaction step such as sulfuric acid, it should be at a strength of between about 75% w/v to about 95% w/v, preferably about 80% w/v to about 90% w/v. The reaction temperature should be about 60° C. to about 130° C., preferably 70° C. to about 120° C. and more preferably about 100° C. to about 120° C. The volume of aqueous acid used for the reaction ranges between about 1-10 volumes, more preferably about 6-9 volumes. The 3-(S)-(+)-(1-carbamoyl-1,1-diphenyl methyl)pyrrolidine of formula XII may be isolated by neutralization with an alkali followed by extraction and isolation of the product, preferably as a foam.

Alternatively, 3-(S)-(+)-(1-carbamoyl-1,1-diphenylmethyl)pyrrolidine of formula XII can be prepared directly from a compound of formula X by reacting it with an aqueous acid such as sulfuric acid, at a strength of between about 75% w/v to about 95% w/v, preferably about 90% w/v to about 95% w/v and most preferably at about 95% w/v. This alternative reaction may occur at a temperature range of about 60° C. to about 100° C., preferably about 65° C. to about 95° C. and more preferably about 75° C. to about 90° C.

The 3-(S)-(+)-(1-carbamoyl-1,1-diphenyl methyl)pyrrolidine of formula XII is then combined with an organic acid such as L(+)-tartaric acid, d-malic acid, 1-malic acid, d-mandelic, 1-mandelic acid or mixtures thereof in a suitable solvent, preferably selected from the group consisting of alcohols, halogenated hydrocarbon, ketones, water or mixtures thereof to form 3-(S)-(+)-(1-carbamoyl-1,1-iphenylmethyl)pyrrolidine salt of formula XIII

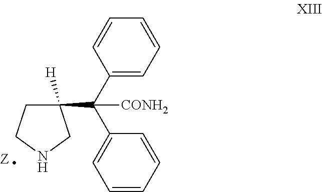

wherein Z is an acid, preferably an organic acid such as L(+)-tartaric acid, d-malic acid, l-malic acid, d-mandelic, l-mandelic acid In one embodiment, the 3-(S)-(+)-(1-carbamoyl-1,1-diphenyl methyl)pyrrolidine of formula XII is dissolved in a suitable solvent such as an alcohol selected from the group consisting of methanol, ethanol, isopropanol or mixtures thereof and L-(+)-tartaric acid is added to form the compound of formula XIII shown in SCHEME III. The addition of the acid to the compound of formula XII to form the 3-(S)-(+)-(1-carbamoyl-1,1-diphenylmethyl)pyrrolidine salt of formula XIII generally occurs at a temperature range of about 20° C. to about 60° C., preferably at a temperature range of about 30° C. to about 55° C., and more preferably at a temperature range of about 40° C. to about 50° C.

Once the 3-(S)-(+)-(1-carbamoyl-1,1-diphenylmethyl)pyrrolidine salt of formula XIII is prepared, it may be reacted with a compound of formula XIV

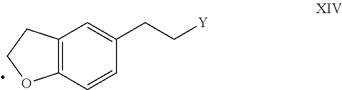

wherein Y is a leaving group preferably selected from the group consisting of I, Cl, Br, brosyl, tosyl, trifluoroacetyl, in a solvent selected from the group consisting of a C₆₋₉ aromatic hydrocarbon, a polar aprotic organic solvent, water and mixtures thereof and an inorganic base to form darifenacin base of formula I. One embodiment of the present invention employs 5-(2-bromoethyl)dihydrobenzofuran as the compound of formula XIV. In this embodiment, the reaction occurs in an aqueous medium in the presence of an inorganic base such as sodium hydroxide, potassium hydroxide, potassium carbonate or mixtures. The reaction forming the darifenacin base from compounds XIII and XIV typically occurs at a reaction temperature range of about 60° C. to about 100° C., preferably about 70° C. to about 100° C. and more preferably about 80° C. to about 100° C.

Before forming the physiologically acceptable salt of darifenacin, one embodiment of the present invention includes a purification step for the darifenacin base. In this purification step, the unreacted 3-(S)-(+)-(1-carbamoyl-1,1-diphenylmethyl)pyrrolidine of formal XIII that is present in the crude darifenacin base is reduced from a level of about 2% to about 5% to a level of about 0.05% to about 0.5%. In one embodiment of this purification step, the crude darifenacin base is converted into its acetate salt by treating the crude darifenacin base with acetic acid and isolating the acetate salt. The acetate salt is subsequently dissolved in water and washed with organic solvents such as toluene, hexane, ethyl acetate or mixtures thereof and then converted to the hydrochloride salt in situ by treating with aqueous hydrochloric acid to a pH of about 1 to about 3. The aqueous solution is then extracted with a suitable solvent such as methylene chloride. The methylene chloride extract is washed with water to remove the unreacted 3-(S)-(+)-(1-carbamoyl-1,1-diphenylmethyl)pyrrolidine. The level of the unreacted 3-(S)-(+)-(1-carbamoyl-1,1-diphenylmethyl)pyrrolidine that is present in the range of about 2% to about 5% is reduced to a level of about 0.05% to about 0.5%.

The crude or purified darifenacin base is then converted to the desired physiologically acceptable salts of darifenacin, such as the hydrobromide of formula II, by dissolving the darifenacin base in a suitable solvent such as acetone and treating the darifenacin solution with the desired pharmaceutically acceptable salt such as an aqueous organic acid or inorganic acid selected from the group consisting of hydrobromic acid, hydrochloric acid, hydroiodidic acid, citric acid, formic acid, maleic acid, acetic acid, succinic acid, tartaric acid, methansulphonic acid or toluenesulphonic acid. Aqueous hydrobromic acid is a preferred pharmaceutically acceptable salt. The addition of the aqueous organic acid or inorganic acid to the darifenacin solution can occur at a temperature range of about 0° C. to about 50° C., preferably about 10° C. to about 40° C. and most preferably about 20° C. to about 30° C. The darifenacin salt thus obtained has a purity of about 98.5% or higher, preferably 99.0% or higher and most preferably 99.5% or higher as measured by HPLC.

A further embodiment of the present invention provides a process for the purification of the desired physiologically acceptable darifenacin salt, by dissolving crude darifenacin salt in a suitable first organic solvent such as methanol, ethanol or isopropanol at a temperature range of about 30° C. to about 90° C., preferably about 45° C. to about 80° C. and most preferably about 60° C. to about 70° C. and treating the solution with a decolorizing agent such as activated carbon. The decolorizing agent is filtered off and the solvent is reduced and/or removed by conventional methods such as distillation from the reaction mixture. The pure darifenacin salt, such as darifenacin hydrobromide, is isolated by adding about 2 to about 15 volumes, preferably about 8 to about 10 volumes, of a suitable second organic liquid such as acetone and isolating the pure darifenacin salt, such as darifenacin hydrobromide, having HPLC purity of about 99.7% or higher.

The crude darifenacin salt can alternatively be purified by dissolving it in about 10 to about 16 volumes, and more preferably about 13 to about 14 volumes, of water at about 75° C. to about 100° C., preferably about 80° C. to about 100° C. and most preferably about 90° C. to about 100° C., followed by treatment with a decolorizing agent. The decolorizing agent is filtered off and the clear filtrate is cooled to a temperature range of from about 10° C. to about 50° C., and more preferably from about 20° C. to about 30° C., and the product obtained is filtered to obtain the desired darifenacin salt, such as darifenacin hydrobromide, having HPLC purity of about 99.7% or higher.

### EXAMPLES

### Example 1

### Preparation of 1-tertiary butyloxy carbonyl-3-(S)-sulfonyloxy pyrrolidine (IX)

100 gms (0.53 moles) of S-(+)-N—BOC-3-hydroxy pyrrolidine was suspended in 500 ml of toluene. 5.0 gms of tetra butyl ammonium bromide and 122 gms (0.64 moles) of p-toluene sulphonyl chloride were added to the reaction mixture at 25° C.-30° C. A solution of 53 gms (1.33 moles) of sodium hydroxide in 123 ml water was slowly added to the reaction mixture in about 90 minutes. The reaction mixture was then heated to 50° C.-60° C. and stirred at this temperature for 4 hours. 300 ml of water and 100 ml toluene were added to the reaction mixture at 50° C.-60° C. The reaction mixture was cooled to about 25° C.-30° C., and the toluene layer was separated from the aqueous layer. The toluene extract was washed with 300 ml water and then dried over anhydrous sodium sulfate. The toluene layer was then concentrated under vacuum maintaining temperature below 70° C. 100 ml of hexane was added to the reaction mass and the reaction mixture was concentrated under vacuum to get an oily residue. 500 ml hexane was added to the reaction mass under stirring. The reaction mass was further stirred for 6 hours at 20° C.-30° C. The solids obtained were filtered and washed with 200 ml of hexane. The product was dried at 40° C. to get 150 gms of 1-tertiary butyloxy carbonyl-3-(S)-sulfonyloxy pyrrolidine as off-white crystals with the following characteristics:

Melting range=56° C.-63° C.

S.O.R.=+11.6°

HPLC Purity >99%.

### Example 2

### Preparation of (S)-2,2-diphenyl-2-[(1-tertiarybutyloxycarbonyl)-3-pyrrolidinil]acetonitrile (X)

15 gms of tetrabutylammonium bromide was added to 300 ml of 50% aqueous sodium hydroxide solution under stirring at 25-35° C. Diphenylacetonitrile, 56.6 gms (0.29 moles) was added to the reaction mixture and stirred at 25° C.-35° C. for 15 minutes. 1-tertiary butyloxy carbonyl-3-(S)-sulfonyloxy pyrrolidine, 100 gms (0.293 moles), was added to the reaction mixture. The reaction mixture was then heated to 90° C.-95° C. and stirred at this temperature for 15 to 30 minutes. 200 ml toluene and 500 ml water was added and the reaction mass was quickly cooled to about 30° C. to 40° C. and stirred at this temperature for 30 minutes. The reaction mass was allowed to settle and the layers were separated. The aqueous layer was back extracted twice with 300 ml toluene. The toluene extracts were combined together and washed twice with 500 ml water. The toluene extracts was dried over anhydrous sodium sulfate. The reaction mass was then concentrated under vacuum to get an oily mass. 200 ml of hexane was added to the oily mass and heated to reflux. The reaction mixture was stirred at the reflux for 1 hour. The reaction mixture was further cooled to 20° C. to 30° C. and stirred for 30 minutes. The solids obtained were filtered and washed with 300 ml hexane. The wet product was dried at 40° C.-50° C. to get 80 gms of (S)-2,2-diphenyl-2-[(1-tertiary butyloxy carbonyl)-3-pyrrolidinil]acetonitrile as off-white crystals with the following characteristics:

Melting Point=113° C.-115° C.

S.O.R.=+7.2°

HPLC >98%.

### Example 3

### Preparation of (S)-2,2-diphenyl-2-(3-pyrrolidinil) acetonitrile (XI)

100 gms (0.276 moles) of (S)-2,2-diphenyl-2-[(1-tertiary butyloxy carbonyl)-3-pyrrolidinil]acetonitrile was dissolved in 100 ml trifluoroacetic acid under stirring, maintaining temperature between 25° C. and 45° C. The reaction mixture was cooled to 20° C.-30° C. and stirred at this temperature for 1 hour. 1 liter of water was added slowly to the reaction mixture and stirred for 10 minutes. The reaction mixture was then cooled to 5° C.-15° C. The pH of the reaction mixture was adjusted to 11-12 using 50% aqueous sodium hydroxide solution and maintaining temperature below 30° C. The reaction mass was then extracted with 1 liter of methylene dichloride (MDC) thrice. All the MDC extracts were combined together and washed with 1 liter of water twice. The MDC extract was dried over anhydrous sodium sulfate and then concentrated under vacuum below 40° C. to remove MDC completely. The reaction mass is degassed for 30 minutes under vacuum at 40° C. to obtain (S)-2,2-diphenyl-2-(3-pyrrolidinil) acetonitrile as oil with the following characteristics:

Yield=70 gms

S.O.R.=+17°

HPLC Purity >98%.

### Example 4

### Preparation of 3-(S)-(+)-(1-carbamoyl-1,1-diphenylmethyl)pyrrolidine tartrate (XIII)

(S)-2,2-diphenyl-2-(3-pyrrolidinil) acetonitrile 100 gms (0.38 moles), was dissolved in 800 ml of 90% w/w sulfuric acid solution under stirring. The reaction mixture was heated to attain a temperature of 100° C.-110° C. The reaction mixture was stirred at 100° C.-110° C. for about 6 hours. The reaction mixture was cooled to about 25° C.-35° C. and slowly quenched into 4 L of ice cold water maintaining temperature below 35° C. 1 liter MDC was added to the reaction mixture and stirred for 15 minutes at 25° C.-35° C. The layers were separated and the MDC layer was discarded. The aqueous layer was cooled to 10° C.-15° C. The pH of the aqueous layer was slowly adjusted to 11-12 using 50% aqueous sodium hydroxide solution. The reaction mixture was extracted thrice with 1-5 L MDC. All the MDC extracts were combined and washed twice with 1-5 L water. The MDC extract was dried over anhydrous sodium sulfate and concentrated under vacuum maintaining a temperature below 45° C. to obtain an 3-(S)-(+)-(1-carbamoyl-1,1-diphenylmethyl)pyrrolidine (XII) as oily residue (75 gms).

The oily residue was dissolved in 75 ml methanol. A solution of 40.17 gms (0.267 mole) L-(+)-tartaric acid in 150 ml of methanol was then slowly added to the reaction mixture under stirring maintaining temperature below 50° C. The reaction mixture was stirred at 40° C.-50° C. for 15 minutes and then cooled to 20° C.-30° C. and stirred at this temperature for 3 hrs. The reaction mixture was further cooled at 5° C.-10° C. and stirred for 1 hour. The product obtained was filtered and washed with 50 ml chilled methanol. The wet product was dried at 40° C. to obtain 80 gms 3-(S)-(+)-(1-carbamoyl-1,1-diphenylmethyl)pyrrolidine tartrate as white crystals with the following characteristics:

Melting range=181° C.-186° C.

S.O.R.=+17°

HPLC purity >98%

### Example 5

### Alternate preparation of 3-(S)-(+)-(1-carbamoyl-1,1-diphenylmethyl)pyrrolidine (XII) from (S)-2,2-diphenyl-2-[(1-tertiary butyloxy carbonyl)-3-pyrrolidinil]acetonitrile (X)

(S)-2,2-diphenyl-2-[(1-tertiarybutyloxycarbonyl)-3-pyrrolidinil]acetonitrile, 100 gm (0.276 moles), was suspended in 500 ml of 95% sulfuric acid. The reaction mixture was slowly heated to about 110° C. and maintained at this temperature under stirring for 8 hours. The reaction mass was then cooled to about 50° C. and slowly quenched into 4 liters of ice-cold water. The reaction mass was extracted twice with 2 liter MDC at 25° C. to 30° C. The upper aqueous layer was separated and the pH of the aqueous layer was adjusted to about 10-12 using 50% aqueous sodium hydroxide solution maintaining temperature below 30° C. The reaction mass was extracted thrice with 2 liter MDC. All the MDC extracts were combined together and washed thrice with 2 liter water and then with 2 liter brine solution. The MDC extract was dried over anhydrous sodium sulfate and concentrated under vacuum maintaining temperature below 40° C. to get 50 gm of 3-(S)-(+)-(1-carbamoyl-1,1-diphenylmethyl)pyrrolidine (XII) as a foam. This can be converted to the tartrate salt of formula XIII, as per Example 4.

### Example 6

### Preparation of Crude Darifenacin Hydromide (II)

100 gms (0.23 moles) 3-(S)-(+)-(1-carbamoyl-1,1-diphenylmethyl)pyrrolidine tartrate was suspended in 300 ml water under stirring at 20° C.-30° C. Powdered potassium carbonate 160.46 gms (1.16 moles) was then added to the reaction mixture. 5-(2-bromoethyl) dihydrobenzofuran, 60.71 gms (0.267 moles) was added to the reaction mixture and heated to about 80° C.-100° C. The reaction mixture was stirred maintaining the temperature at 80° C.-100° C. for about 1 to 2 hours. The reaction mixture cooled rapidly to attain a temperature of about 40° C.-50° C. Toluene 300 ml was added to the reaction mixture, cooled to 20° C.-30° C. and stirred for 30 minutes. The layers were separated and the aqueous layer was extracted twice with 300 ml toluene. All the toluene extracts were combined together, and the product was extracted in 400 ml of 20% aqueous acetic acid solution thrice. The combined acetic acid extracts were washed twice with 200 ml toluene. The pH of the acetic acid was adjusted to about 1-2 using 20% hydrochloric acid solution. The reaction mass was extracted with 400 ml MDC thrice. All the MDC extracts were combined together and washed with 300 ml 20% hydrochloric acid solution and then with 300 ml water. The reaction mixture was further washed with 300 ml of saturated aqueous solution sodium bicarbonate twice and then with 300 ml of water. The reaction mass was dried over anhydrous sodium sulfate and concentrated under vacuum maintaining temperature below 40° C. to obtain oily residue. Acetone 500 ml was added to the oily residue and maintained at 20° C.-30° C. for 15 minutes. 38 gms of 48% aqueous HBr solution was added to the reaction mixture at 20° C.-30° C. The reaction mixture was further stirred at 20° C.-30° C. for 2 hrs. The precipitated product was filtered and washed with 300 ml chilled acetone. The wet product was dried at 40° C.-45° C. under vacuum to obtain 95 gms of crude darifenacin hydrobromide as white crystals with an HPLC Purity >98.5%.

### Example 7

### Purification of Crude Darifenacin Hydrobromide

95 gm of crude darifenacin hydrobromide obtained in Example 5 was suspended in 475 ml methanol. The reaction mass was heated to reflux at about 60° C.-70° C. to get a clear solution. Activated charcoal 0.2 gm was added to the reaction mixture and stirred, maintaining the reflux for 15 minutes. The reaction mixture was then filtered hot over a hyflo bed and the hyflo bed was washed with 95 ml methanol. The clear filtrate was then concentrated under vacuum maintaining temperature below 40° C. Acetone 950 ml was added and the reaction mixture cooled to 20° C.-30° C. and stirred at this temperature for 1 hour. The product obtained was filtered and washed with 25 ml chilled acetone. The wet product was dried at 50° C. under vacuum to obtain 85.5 gms darifenacin hydrobromide with the following characteristics:

S.O.R.=+46°

HPLC purity >99.7%.

### Example 8

### Purification of Crude Darifenacin Hydrobromide

100 gm of crude darifenacin hydrobromide was suspended in 1300 ml water. The suspension was heated to reflux at 90° C.-100° C. to get a clear solution. 2 gm activated charcoal was added, and the reaction mixture was stirred under reflux for 30 minutes. The reaction mixture was filtered hot over a hyflo bed. The clear filtrate was gradually cooled under stirring to attain 20° C.-30° C. temperature. The reaction mass was stirred at this temperature for 2 hours. The product obtained was filtered and washed with 100 ml water. The wet material was dried at 40° C.-50° C. under vacuum to obtain 80 gm of pure darifenacin hydrobromide as white crystals with the following characteristics:

HPLC purity >99.7%

S.O.R=+46°

The invention illustratively described herein suitably may be practiced in the absence of any element or elements, limitation or limitations which is not specifically disclosed herein. Thus, for example, in each instance herein any of the terms “comprising,” “consisting essentially of” and “consisting of” may be replaced with either of the other two terms. The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A compound of formula 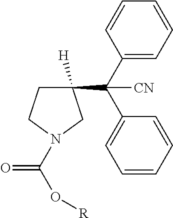
wherein R is linear or branched C1-10 alkyl, phenyl, tolyl, ortho-, meta- or para-xylyl.

2. A process for the manufacture of a compound of formula X 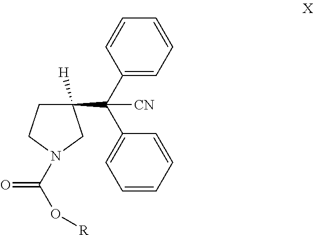
wherein R is linear or branched C1-10 alkyl, phenyl, tolyl, ortho-, meta- or para-xylyl comprising the steps of: a) reacting a compound of formula VIII 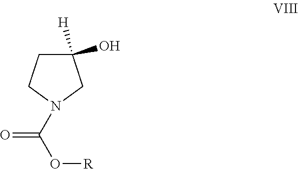
wherein R is linear or branched C1-10 alkyl, phenyl, tolyl, ortho-, meta- or para-xylyl, with a sulfonyl halide in the presence of a phase transfer catalyst and an inorganic base to give a compound of formula IX 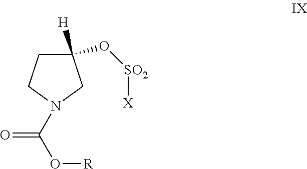
wherein R is linear or branched C1-10 alkyl, phenyl, tolyl, ortho-, meta- or para-xylyl and X is linear or branched C1-1O alkyl, phenyl, tolyl, ortho-, meta- or para-xylyl; and b) reacting a compound of formula IX with diphenylacetonitrile and an inorganic base to give the compound of formula X.

3. The process of claim 2, wherein the conversion of compound of formula VIII to the compound of formula IX is carried out in a biphasic reaction medium comprising water and one or more organic solvents, in the presence of one or more alkali metal salts.

4. The process of claim 3, wherein the organic solvent is selected from the group consisting of toluene, xylene, tetrahydrofuran and mixtures thereof.

5. The process of claim 3, wherein the one or more alkali metal salts is selected from the group consisting of sodium hydroxide, potassium hydroxide, potassium carbonate and mixtures thereof.

6. The process of claim 2, wherein the conversion of the compound of formula IX to the compound of formula X is carried out in an aqueous solution of one or more inorganic bases in the presence of one or more phase transfer catalysts.

7. The process of claim 6, wherein the one or more inorganic bases is selected from the group consisting of sodium hydroxide, potassium hydroxide, potassium carbonate, sodium bicarbonate and mixtures thereof.

8. The process of claim 6, wherein the one or more phase transfer catalysts is selected from the group consisting of tetrabutylammonium bromide, tetrabutylammonium iodide, tetrabutylammonium hydroxide and mixtures thereof.

9. The process of claim 6, wherein the aqueous solution of the one or more inorganic bases has a concentration between about 25% to about 70% w/v.

10. The process of claim 6, wherein the reaction is carried out at the temperature range of between about 60° C. to about 110° C.

11. A process for manufacturing darifenacin from a compound of formula X 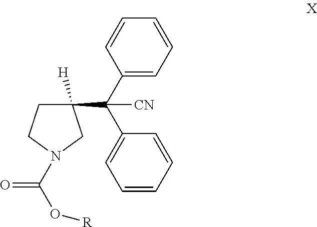
wherein R is linear or branched C1-10 alkyl, phenyl, tolyl, ortho-, meta- or para-xylyl comprising the steps of:
(a) converting a compound of formula X to 3-(S)-(+)-(1-carbamoyl-1,1-diphenylmethyl)pyrrolidine of formula XII 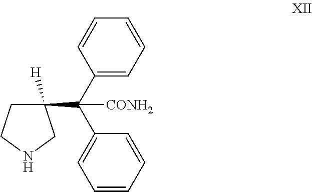
(b) reacting 3-(S)-(+)-(1-carbamoyl-1,1-diphenylmethyl)pyrrolidine of formula XII with an organic acid to produce 3-(S)-(+)-(1-carbamoyl-1,1-diphenylmethyl)pyrrolidine salt of formula XIII 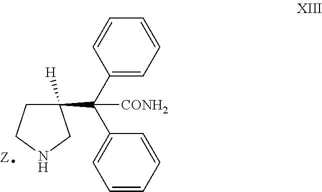
wherein Z is an acid;
(c) reacting 3-(S)-(+)-(1-carbamoyl-1,1-diphenylmethyl)pyrrolidine salt of formula XIII and a compound of formula XIV 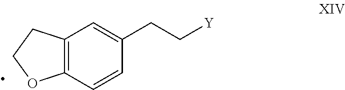
wherein, Y is a leaving group, to obtain darifenacin base.

12. The process of claim 11 wherein the compound of formula X is converted to the compound of formula XII by reacting a compound of formula X with a deprotecting agent to obtain (S)-2,2-diphenyl-2-(3-pyrrolidinil)acetonitrile of formula XI 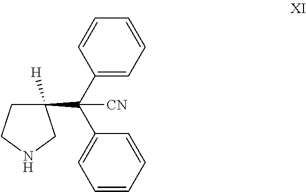
and reacting (S)-2,2-diphenyl-2-(3-pyrrolidinil)acetonitrile of formula XI with a hydrolyzing agent to obtain 3-(S)-(+)-(1-carbamoyl-1,1-diphenylmethyl)pyrrolidine of formula XII.

13. The process of claim 11 wherein the compound of formula X is converted to the compound of formula XII by reacting a compound of formula X with a strong acid to give 3-(S)-(+)-(1-carbamoyl-1,1-diphenylmethyl)pyrrolidine of formula XII.

14. The process of claim 11 further comprising the step of reducing the amount of 3-(S)-(+)-(1-carbamoyl-1/1-diphenylmethyl)pyrrolidine in the darifenacin base to about 0.05% to about 0.5%.

15. The process of claim 14 wherein the reduction comprises the steps of: i. reacting darifenacin base with acetic acid to from darifenacin acetate salt; ii. adding aqueous hydrochloric acid to the solution of step i to form darifenacin hydrochloride salt; iii. extracting darifenacin from the solution of step ii with a suitable solvent; and iv. washing the extract of step iii with water to reduce the amount of 3-(S)-(+)-(1-carbamoyl-1,1-diphenylmethyl)pyrrolidine in the resulting darifenacin base to about 0.05% to about 0.5%.

16. The process of claim 11 further comprising the step of reacting the darifenacin base with an acid selected from the group consisting of hydrobromic acid, hydrochloric acid, hydroiodidic acid, citric acid, formic acid, maleic acid, acetic acid, succinic acid, tartaric acid, methansulphonic acid and toluenesulphonic acid to form a darifenacin salt.

17. The process of claim 16 wherein the darifenacin salt is darifenacin hydrobromide.

18. The process of claim 16 further comprising a purification step for the darifenacin salt comprising: a. dissolving the darifenacin salt in an organic solvent; b. filtering the solution of step a; c. concentrating the filtrate of step b; d. adding a second organic solvent to the concentrated of step c; and e. isolating pure darifenacin salt having an HPLC purity of about 99.7% or higher.

19. The process of claim 16 further comprising a purification step for the darifenacin salt comprising: i) dissolving the darifenacin salt in water; ii) filtering the water solution of step a; and iii) isolating pure darifenacin salt having an HPLC purity of about 99.7% or higher.

20. A darifenacin salt prepared by the process of claim 16.
